(19) United States
(12) Patent Application Publication
(10) Pub. No.: US 2026/0273299 A1
(43) Pub. Date: Sep. 17, 2026

(54) COMPACT WEARABLE PULSED ELECTROMAGNETIC FIELD THERAPY SYSTEM FOR BONE HEALING WITH IMPLANT FIELD CONCENTRATION

(71) Applicant: Wurth Medical Innovations, LLC, Brentwood, TN (US)

(72) Inventors: Todd R. Wurth, Brentwood, TN (US); Barry R. Hix, Birmingham, AL (US); Thomas J. Bachinski, Lakeville, MN (US)

(73) Assignee: Wurth Medical Innovations, LLC, Brentwood, TN (US)

(21) Appl. No.: 19/680,504

(22) Filed: May 18, 2026

Related U.S. Application Data
(63) Continuation-in-part of application No. 19/566,734, filed on Mar. 13, 2026.
(63) Continuation-in-part of application No. 19/202,837, filed on May 8, 2025.
(60) Provisional application No. 63/644,868, filed on May 9, 2024.

(51) Int. Cl. A61N 2/00; A61F 2/44; A61N 2/02; A61N 2/06

(52) U.S. Cl. CPC A61N 2/004; A61F 2/4455; A61N 2/02; A61N 2/06

(57) ABSTRACT

Disclosed herein is a compact wearable pulsed electromagnetic field (PEMF) therapy system. The system may be used for bone healing and optimized for activation of implantable passive electromagnetic components. The system may comprise an external PEMF generator having a compact electromagnetic coil, pulse generation circuit, and wearable housing. In one application, the generator operates in implant-activation mode (about 3-10 mT) to activate implanted passive components —permanent magnets, with or without integrated electromagnetic induction coils, integrated into standard orthopaedic hardware —positioned directly at a bone fracture site. Advantageously, the system may eliminate geometric field divergence inherent in conventional circumferential external PEMF systems, enabling 40-60% coil diameter reduction and 60-85% weight reduction while achieving targeted field delivery at the fracture site. The system may also be architecturally configured for a standalone PEMF generation mode (about 8-20 mT) independent of implanted components, which may have application as a clinical bone growth stimulator.

### CROSS-REFERENCE TO RELATED APPLICATIONS

This application claims priority to U.S. Provisional Application No. 63/644,868, filed May 9, 2024, U.S. Non-Provisional application Ser. No. 19/202,837, filed May 8, 2025, titled “Medical Hardware Energy Delivery System,” and U.S. Non-Provisional application Ser. No. 19/566,734, filed Mar. 13, 2026, titled “Compact Wearable Pulsed Electromagnetic Field Therapy System for Bone Healing with Implant Field Concentration.” The entire disclosures of U.S. Provisional Application No. 63/644,868 and U.S. Non-provisional Application Nos. Ser. No. 19/202,837 and Ser. No. 19/566,734 are hereby incorporated herein by reference.

### FIELD OF INVENTION

The present invention relates generally to electromagnetic therapy systems for bone healing, and more particularly, to compact, wearable pulsed electromagnetic field (PEMF) generators, which may be optimized for use with implantable passive electromagnetic components, and to dual-mode PEMF systems, which may be capable of operating both as implant activators and as standalone PEMF generators.

### BACKGROUND

Bone fractures represent a significant healthcare burden, with approximately 6-8 million fractures occurring annually in the United States alone. While most fractures heal with standard immobilization or surgical fixation, 5-30% of cases experience delayed healing or nonunion, particularly in high-risk patient populations including the elderly, diabetics, smokers, and patients with osteoporosis or compromised vascular supply.

Electromagnetic field therapy has been established as an effective adjunctive treatment for bone healing for over 45 years. Two primary modalities exist:
(a) Pulsed Electromagnetic Field (PEMF) Therapy: External devices generate time-varying magnetic fields that induce electric currents in bone tissue via electromagnetic induction (Faraday's law). PEMF devices have been FDA-cleared since 1979 (e.g., Physio-Stim K 881658, OrthoGen K944782) for treatment of fresh fractures, delayed unions, and nonunions.
(b) Capacitively Coupled Electric Field (CCEF) Therapy: Devices generate electric fields in tissue through capacitive coupling. CCEF therapy has been FDA-cleared since 1984 for spinal fusion and nonunion treatment (e.g., EBI Bone Healing System K033522).

Both PEMF and CCEF therapies have demonstrated efficacy in multiple randomized controlled trials, with clinical evidence showing 10-30% improvement in union rates and 2-4 week reductions in healing time compared to controls.

### Conventional External PEMF Devices—Design Characteristics

Current FDA-cleared external PEMF bone growth stimulators (Physio-Stim, OrthoGen, Orthopak) employ a circumferential coil architecture in which the treatment coil must be centered around the fracture site. The device instructions for use language for each cleared device explicitly states that the coils must be centered around the fracture site to be effective, and Orthofix markets the Physio-Stim's 360-degree field coverage as a device feature. This circumferential architecture requires:
Large electromagnetic coils (15-25 cm diameter) to encircle the treatment anatomy
High power consumption (20-50 watts) to achieve adequate field strength at depth when field energy is distributed circumferentially
Heavy, bulky form factors (2-4 Kg total weight)
Wearable form factors that remain large and bulky due to the circumferential coil architecture requirement, limiting patient acceptance and comfort during extended daily wear
Size and weight (2-4 kg total) that create practical barriers to consistent daily wear compliance

These design constraints result in poor patient compliance. Published literature reports compliance rates of only 60-80% with conventional external PEMF devices, with non-compliance primarily attributed to device size, weight, discomfort during extended wear, and the circumferential positioning requirement which limits practical use in daily activities.

### Conventional Implantable CCEF Devices—Limitations

Active implantable CCEF devices (EBI Bone Healing System, SpinalPak) address compliance issues but introduce different limitations, such as:
Battery implantation requiring surgical dissection
Battery removal surgery at 6-9 months post-implantation
Infection risk at battery pocket site (1-3% in published literature)
Skin erosion over battery packs in thin patients
Patient anxiety regarding implanted electronics

Thus, opportunities exist for further improvements and advancements in such devices to enhance functionality and patient compliance.

### BRIEF SUMMARY

Incorporated herein are energy delivery systems that are operable to generate and deliver magnetic and/or electric fields to a target area proximate to an implantation site. Such systems may include implantable passive electromagnetic components comprising stacked neodymium magnets, with or without integrated electromagnetic induction coils, housed within a biocompatible encapsulation and integrated into standard orthopaedic hardware.

The implantable passive electromagnetic components may operate through, at least, two mechanisms when exposed to external PEMF fields:
(1) Positioning electromagnetic therapy delivery directly at the fracture site, eliminating geometric field divergence inherent in external-only PEMF systems where the therapeutic coil is positioned 5-10 cm from the fracture site and
(2) Electromagnetic induction generating capacitively coupled electric fields (CCEF) via Faraday induction, delivering combined PEMF+CCEF therapy without requiring implanted batteries or active electronics.

Disclosed herein is an extension of this system by providing external PEMF generator designs, which may be optimized to harness the full therapeutic potential of implanted components positioned directly at fracture sites.

External PEMF generators may be the energy source for activating the implanted passive components. The external PEMF generators may be specifically optimized for use with implanted passive components, which may enable compact wearable form factors. Conventional PEMF devices require large circumferential coil geometries and high power to achieve therapeutic field strength at fracture sites 5-10 cm from the external coil. When passive electromagnetic components are positioned directly at fracture sites, these geometric constraints may be replaced by optimized compact designs delivering targeted field energy to the implant site.

Disclosed herein are external PEMF generator designs specifically optimized for use with implanted passive electromagnetic components. These designs take advantage of reduced geometric field divergence requirements to enable compact, truly wearable form factors not achievable with conventional external-only circumferential PEMF systems.

Also disclosed herein are dual-mode PEMF systems. These systems may be capable of operating as activators for patients with implanted passive electromagnetic components (“OsteoCore components”) and/or as standalone PEMF generators for independent operation. Standalone operation as a bone growth stimulator for patients without implanted components represents a future clinical application pathway subject to independent clinical validation and regulatory clearance, as described herein.

### BRIEF DESCRIPTION OF EMBODIMENTS

Disclosed herein are compact wearable pulsed electromagnetic field (PEMF) therapy systems. Said systems may be specifically optimized for use with implantable passive electromagnetic components. Also disclosed herein are dual-mode systems architecturally capable of both implant activation and/or standalone PEMF generation.

In a first aspect, provided herein is a wearable PEMF therapy system. The system may comprise an external PEMF generator including an electromagnetic coil (may have a diameter of about 8-18 cm), pulse generation circuit (may have frequencies of about 1-200 Hz), power source (may have about 2-15 watts), and housing (may have a mass of about 200-1200 grams). Such an embodiment may represent a 40-60% coil diameter reduction, 70-90% power reduction, and 60-85% weight reduction compared to conventional external circumferential PEMF devices, achieved through implant-optimized design eliminating the need for large circumferential coil geometries.

In a second aspect, provided herein is a dual-mode PEMF therapy system. The system may be configured to operate in: (a) an implant-activation mode generating targeted pulsed magnetic fields of about 3-10 mT to activate implanted passive electromagnetic components positioned at a fracture site; and (b) a standalone PEMF generation mode producing pulsed magnetic fields of about 8-20 mT for operation independent of implanted components.

In a third aspect, provided herein is a bone healing monitoring system. The system may utilize implanted permanent magnets as passive sensors. Changes in the impedance of an external measurement coil resulting from tissue magnetic susceptibility changes during fracture healing may enable non-invasive assessment of healing progress without additional implanted sensors, batteries, or active electronics.

In a fourth aspect, provided herein is conformable coil architectures. The architectures may include curved rigid coils, flexible continuous coils on flex-PCB substrates, and segmented articulated coil arrays comprising multiple rigid coil elements connected by flexible electrical conductors, enabling the coil array to conform to anatomies of varying circumference.

### BRIEF DESCRIPTION OF THE DRAWINGS

So that the manner where the above recited features may be understood in detail, a more particular description, briefly summarized above, may be had by reference to example aspects, some of which are illustrated in the appended drawings.

FIG. 1 is a side-by-side comparison diagram illustrating the architectural distinction between a conventional circumferential external PEMF device (left panel) and an embodiment of a compact unilateral OsteoCore-paired PEMF generator of the present invention (right panel), showing relative coil diameter, device weight, and coil placement geometry.

FIG. 2 is a schematic diagram illustrating dual-mode operation of an embodiment of a compact PEMF generator: Panel A shows implant-activation mode with OsteoCore implanted components at the fracture site; Panel B shows standalone PEMF generation mode without implanted components.

FIG. 3 is a perspective view of an embodiment of a compact disc-shaped wearable PEMF generator showing a housing, electromagnetic coil (dashed), elastic strap attachment, user interface buttons, and USB-C charging port.

FIG. 4 is a cross-sectional view of the compact disc-shaped PEMF generator of FIG. 3, showing internal component arrangement including an electromagnetic coil, battery, electronics PCB, housing, and strap attachment points.

FIG. 5 is a schematic diagram of an embodiment of a smartphone-connected PEMF therapy system showing wireless communication between the wearable PEMF generator, paired smartphone application, and cloud-based healthcare provider portal.

FIG. 6 is a cross-sectional diagram of a patient limb illustrating electromagnetic field delivery from an embodiment of a compact external PEMF generator through tissue to OsteoCore implanted passive components at the fracture site, showing field convergence and therapeutic field zone concentration at the implant location.

FIG. 7 is a comparative diagram illustrating the U.S. annual fracture patient population addressable by the OsteoCore-paired implant platform versus the broader platform architecture addressable market including future standalone PEMF generation applications pending clinical validation.

FIG. 8 is a block diagram of the electronic architecture of an embodiment of a compact PEMF generator, showing the microcontroller, power amplification stage, current sensing circuit, battery, voltage regulator, charging circuit, electromagnetic coil, and BLE wireless communication module.

FIG. 9 is a flowchart illustrating a method of delivering and monitoring OsteoCore-paired PEMF therapy, including surgical implantation, post-operative device deployment, compliance logging, remote provider review, and healing-based therapy cessation decision.

FIG. 10 is a schematic diagram of an embodiment of a magnetic field-based bone healing monitoring system, illustrating impedance measurement between the external coil and implanted permanent magnets, magnetometer-based positioning confirmation, and healing stage classification based on inductance changes.

FIG. 11 is a line graph illustrating relative inductance change over time post-surgery, showing three healing stage zones corresponding to soft callus formation, mineralizing callus, and mature bone formation, with threshold values subject to clinical validation.

FIG. 12 is a comparative diagram illustrating three embodiments of conformable coil architectures: Panel A, curved rigid coil; Panel B, flexible continuous coil on flex-PCB substrate; and Panel C, segmented articulated coil array, each shown in flat and limb-wrapped configurations.

FIG. 13 is a detailed view of an embodiment of a segmented articulated coil array showing the overall flat configuration, cross-section of a single coil element, cross-section of an articulation joint, and the device wrapped around a forearm.

FIG. 14 is a diagram illustrating universal sizing capability of a segmented articulated coil array on four limb sizes, showing circumferential coverage ranging from approximately 340 degrees on a small wrist to approximately 100 degrees on a large thigh, with therapeutic field delivery at the OsteoCore implant site in each configuration.

FIG. 15 is a diagram illustrating three selective coil element activation modes for a segmented articulated coil array: Panel A, all-panel activation; Panel B, proximity-based selective activation using magnetometer detection of the OsteoCore implant; and Panel C, sequential scanning activation.

FIG. 16 is a diagram illustrating integration of an embodiment of a conformable PEMF device into a textile compression garment, showing the garment flat with integrated pocket, device insertion, completed assembly worn on the lower leg, and cross-sectional view showing device positioning relative to tissue layers and fracture site.

### DETAILED DESCRIPTION

Disclosed herein are compact external PEMF generator systems. The systems may be specifically designed to activate OsteoCore implanted components. One clinical application of the present invention is activation of OsteoCore implanted components in patients undergoing operative fracture fixation with OsteoCore-enabled hardware. A secondary architectural capability —standalone PEMF generation independent of implanted components —is described herein as a device functionality, which may have application as a clinical bone growth stimulator.

### I. Compact Wearable External PEMF Generator

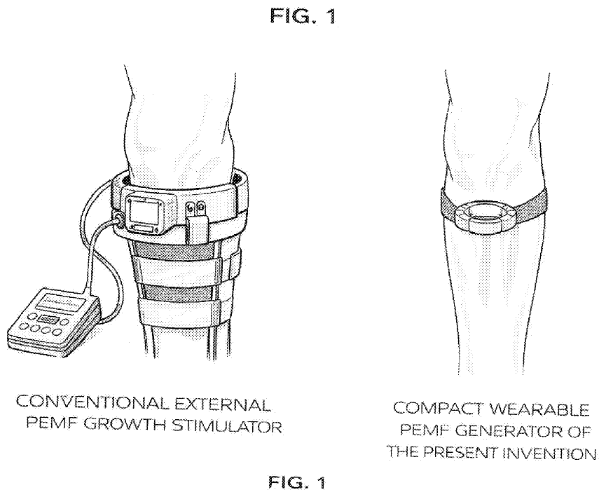

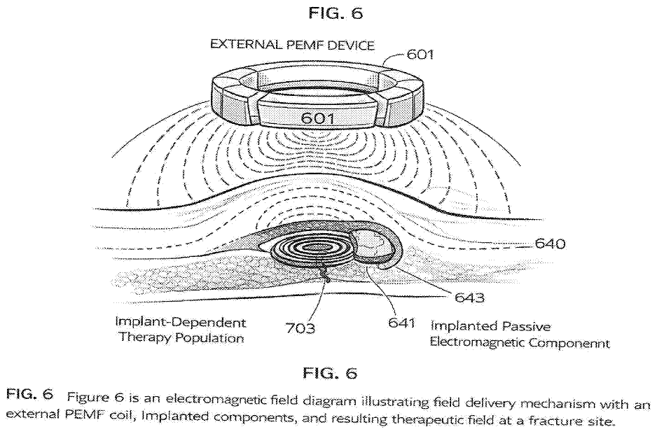

Disclosed herein is a compact wearable external PEMF generator, which may be specifically optimized for activation of OsteoCore implanted passive electromagnetic components. As illustrated in FIG. 1, the architectural distinctions between conventional circumferential PEMF devices and an embodiment of a compact wearable external PEMF generator are shown. Conventional devices require a large coil to encircle the fracture site, while the illustrated embodiment of a compact generator disclosed herein is positioned unilaterally over the OsteoCore implant site. The optimization rationale is as follows: conventional external PEMF devices employ circumferential coil architectures requiring the coil to encircle the fracture site, necessitating large coil diameters (15-25 cm) and high power output (20-50 watts) to achieve adequate field strength at depth. When OsteoCore passive electromagnetic components are positioned directly at the fracture site within the implanted hardware, the external generator need only deliver sufficient field strength at the skin surface to drive the implanted concentrators. The implanted components accomplish the therapeutic field concentration at the fracture site, as shown in FIG. 6. In some embodiments, the compact wearable external PEMF generator comprises the following design parameters:
Coil diameter: about 8 to about 18 cm (40-60% reduction vs. conventional circumferential devices)
Power consumption: about 2 to about 15 watts (70-90% reduction)
Total device mass: about 200 to about 1200 grams (60-85% reduction)
External magnetic field output: about 3 to about 10 mT (implant-activation mode)

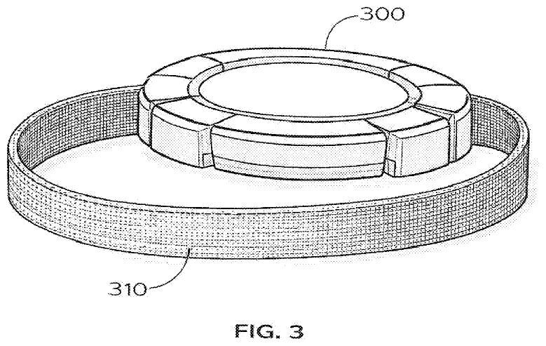

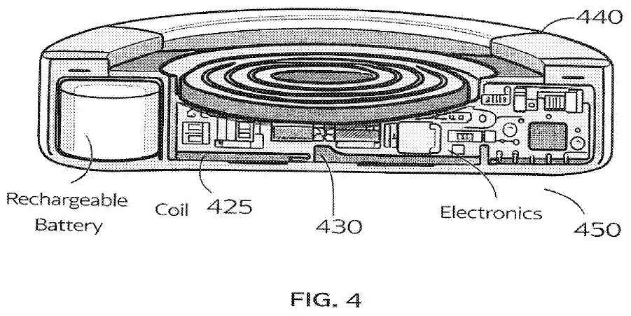

A representative embodiment of the compact wearable PEMF generator is shown in perspective view in FIG. 3. The compact wearable PEMF generator may comprise, for example, one or more of the following: a housing (which may be disc-shaped or any other shape), an electromagnetic coil, an attachment for affixing the housing to a body (which may be an elastic strap attachment), user interface elements, charging port (which may be a USB-C port), and other features. FIG. 4 provides a cross-sectional view of the compact wearable PEMF generator shown in FIG. 3 that shows one embodiment of an internal component arrangement within the housing, which may contain, for example, one or more of the following: electromagnetic coil, a battery pack, and electronics, such as a printed circuit board.

### II. Architecture

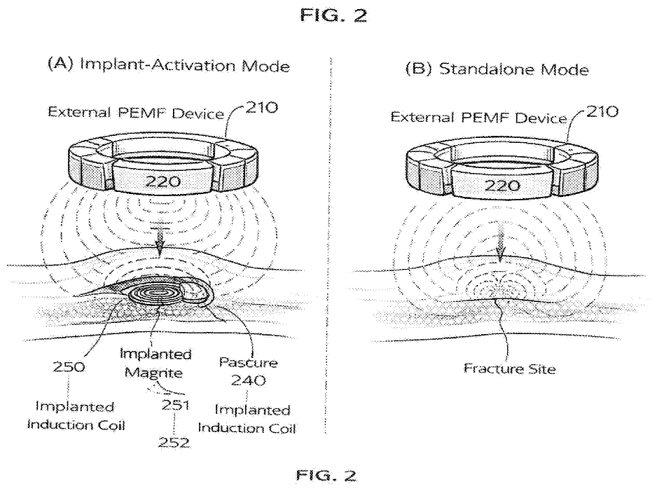

The compact external PEMF generator may be configured to operate in any number or variety of modes. In one embodiment, such as that illustrated schematically in FIG. 2, the compact external PEMF generator may be configured to operate in two modes. The two modes may be an implant-activation mode and a standalone PEMF generation mode. Descriptions of the two exemplary modes are provided below:
Mode A—Implant-Activation Mode: The generator may operate at about 3-10 mT external field output, about 2-8 watts power consumption, and with about a 24-hour battery life per charge cycle. This mode may be designed for patients with OsteoCore implanted components, where the external generator activates the passive concentrators positioned at the fracture site. Clinical performance in this mode is consistent with published large animal model data.
Mode B—Standalone PEMF Generation Mode: The generator may be architecturally capable of operating at about 8-20 mT external field output, about 5-15 watts power consumption, and may not require implanted OsteoCore components. This mode represents a hardware capability of the device. This mode may advantageously stimulate bone growth, even independent of OsteoCore implanted components.

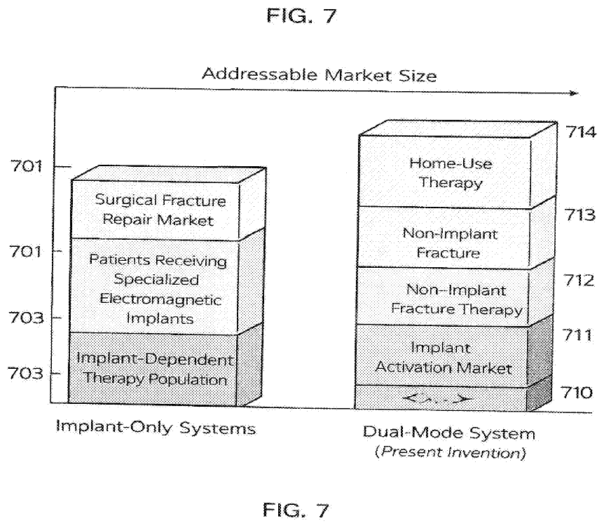

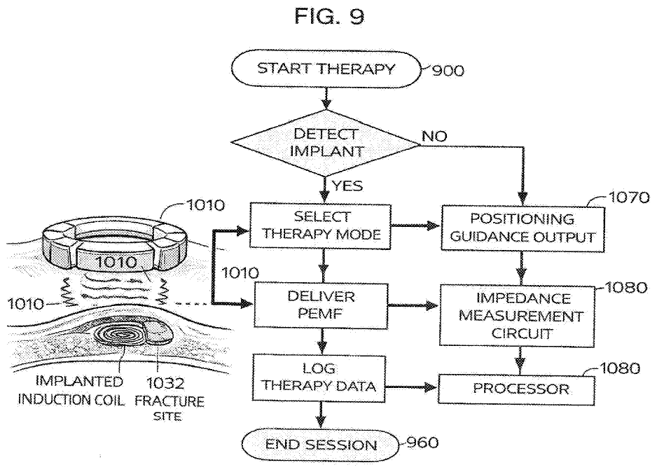

In the dual-mode embodiment, the architecture provides engineering flexibility enabling a single device platform to serve the primary OsteoCore-paired indication while preserving optionality for clinical applications through the standalone generation capability. FIG. 7 illustrates the comparative addressable patient population between the OsteoCore-paired platform and the dual-mode architecture embodiment. FIG. 9 is a flowchart illustrating an embodiment of the OsteoCore-paired therapy delivery and monitoring method, which is also envisioned herein.

### III. Bone Healing Monitoring System

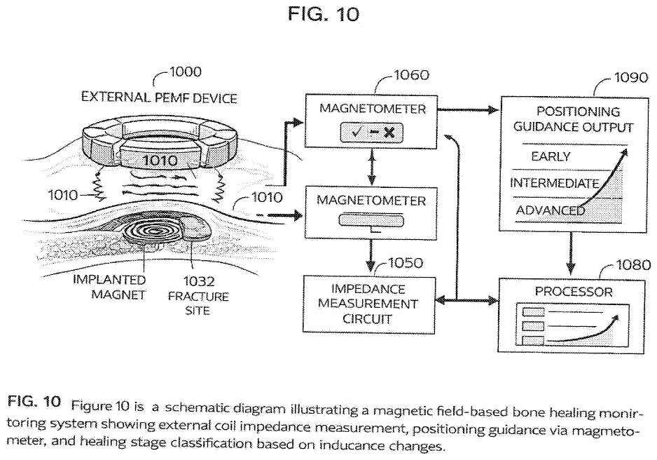

Disclosed herein is a bone healing monitoring system. The bone healing monitoring system may utilize implanted OsteoCore permanent magnets as passive sensors, such as the embodiment illustrated in FIG. 10.

In one embodiment, the monitoring system comprises the following steps:
(a) The external coil applies a low-amplitude interrogation signal at a frequency distinct from the therapeutic PEMF signal.
(b) The impedance of the external coil is measured during the interrogation signal. Coil impedance is influenced by the magnetic coupling between the external coil and the implanted permanent magnets, which in turn is modulated by the magnetic susceptibility of the surrounding tissue.
(c) As fracture healing progresses, tissue magnetic susceptibility changes: early-stage soft callus has different magnetic properties than intermediate mineralizing callus and advanced mature bone. These changes produce measurable changes in external coil impedance over the healing timeline.
(d) Healing stage classification is derived from the measured impedance change relative to baseline: Early healing stage (<0.5% ΔL/L₀), Intermediate healing stage (0.5-1.0% ΔL/L₀), Advanced healing stage (>1.0% ΔL/L₀).

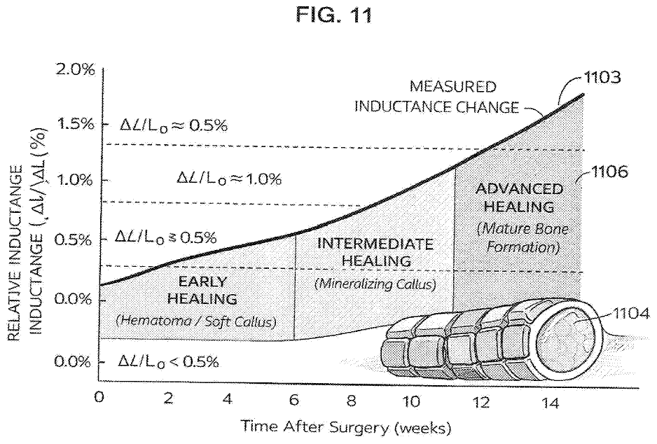

The impedance threshold values recited herein represent initial engineering estimates, as shown graphically in FIG. 11. Specific threshold values for clinical use will be vary and may be refined through clinical validation studies correlating external coil impedance measurements with radiographic healing scores (e.g., RUST scale) in human subjects.

### IV. Coil Architectures

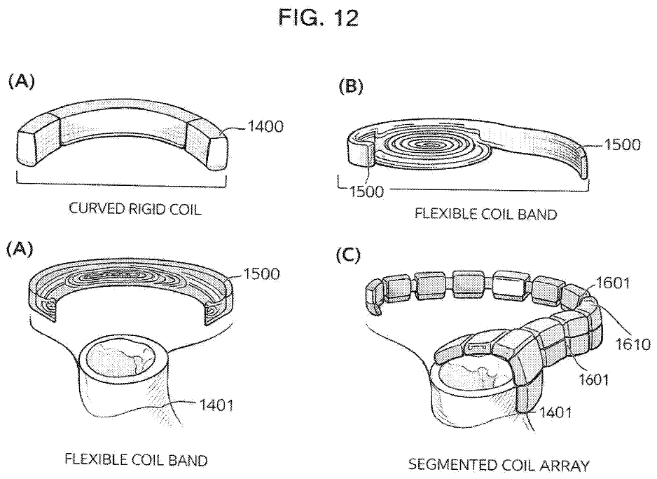

Disclosed herein are coil architectures. The coil architectures may adapt to a patient's body, including but not limited to the curved external anatomy of a patient's limb. The architectures may enable consistent therapeutic positioning across anatomical variations, as illustrated by the comparative embodiments of FIG. 12. Advantageously, such designs may be comfortable for the patient and, thereby, increase patient use and satisfaction.

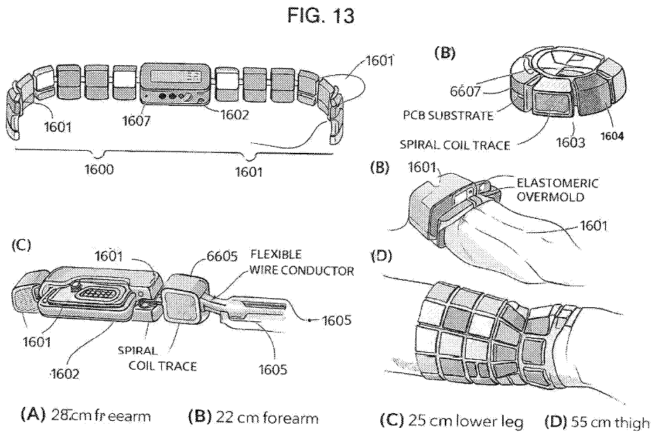

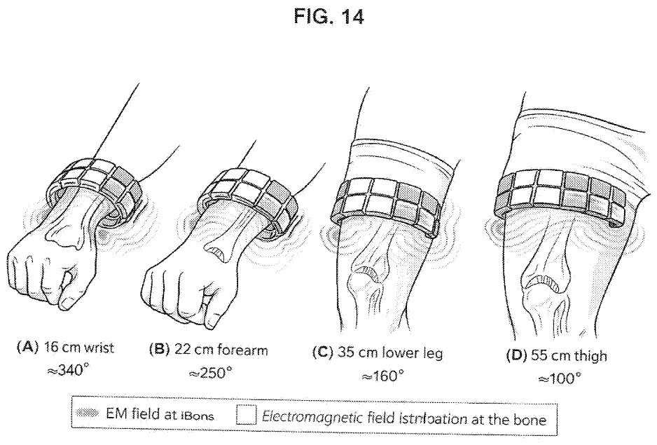

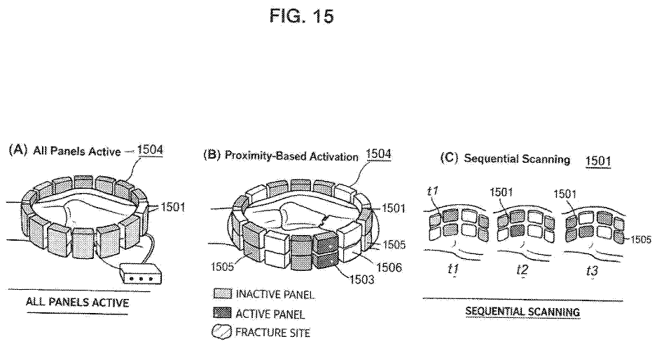

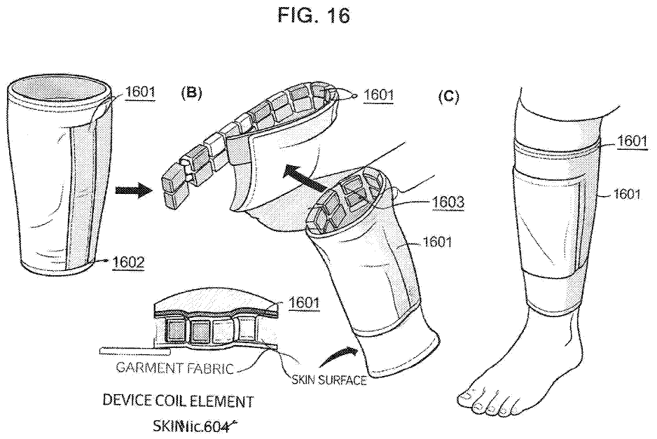

Three exemplary embodiments are detailed below:
Architecture A—Curved Rigid Coil: A rigid coil housing having a concave patient-contact surface with radius of curvature between about 3 cm and about 15 cm, enabling conformal fit against rounded limb surfaces. This architecture is illustrated in FIG. 12, Panel A.
Architecture B—Flexible Continuous Coil (flex-pcb): an Electromagnetic Coil Fabricated on a flexible printed circuit board (which may be any substrate, e.g., polyimide or liquid crystal polymer substrate, about 50 to about 200 μm thickness) configured as an elongated band capable of wrapping around limb circumferences from about 15 cm to about 65 cm. This architecture is illustrated in FIG. 12, Panel B.
Architecture C—Segmented Articulated Coil Array: A plurality of discrete rigid coil elements (about 6-16 elements, each about 2×3 cm to about 4×6 cm) connected by flexible electrical conductors providing both electrical continuity and mechanical articulation between adjacent elements. Cumulative articulation across multiple joints enables the array to conform to limb circumferences from about 15 cm to about 55 cm. Selective activation of individual coil elements based on magnetometer-detected proximity to implanted components optimizes field delivery efficiency. This architecture is illustrated in FIG. 12, Panel C, with detailed structural views provided in FIG. 13. FIG. 14 illustrates universal sizing capability across four representative limb circumferences. FIG. 15 illustrates three selective coil activation modes. FIG. 16 illustrates integration of the conformable device into a textile compression garment.

### V. Electronic Architecture and Connectivity

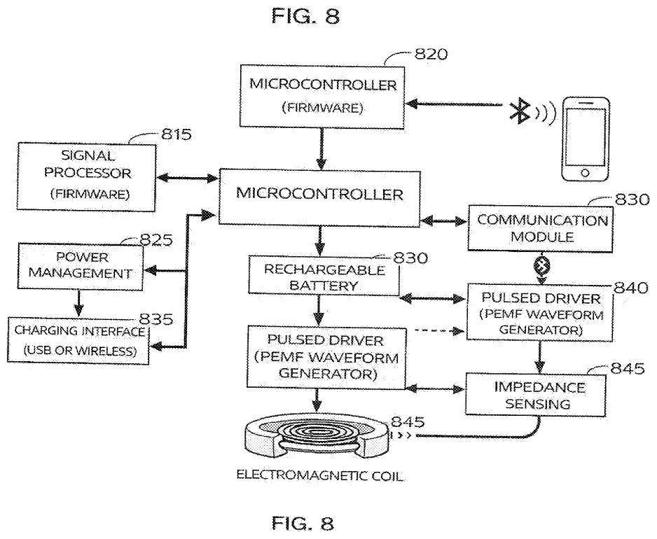

The electronic architecture of the compact PEMF generator may comprise any number of components (e.g., coils, electronics, and so on). In one embodiment and as illustrated in the block diagram of FIG. 8, the electronic architecture comprises: microcontroller unit (MCU) for signal generation and device management; power amplification stage driving the electromagnetic coil; current sensing circuit for real-time output monitoring; a battery (e.g., a rechargeable lithium-ion battery (about 8-20 Wh capacity)); charging circuit (e.g., with a USB-C interface); and a communication module (e.g., a Bluetooth Low Energy (BLE) wireless communication module).

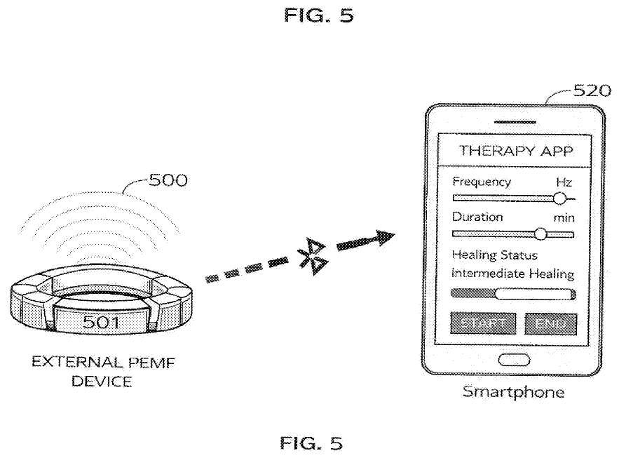

In embodiments having a communication module (such as a BLE module), the module enables bidirectional communication with, for example, a paired smartphone application, as illustrated in FIG. 5. This offers may benefits, such as a compliance tracking with daily therapy hour logging; therapy progress visualization; remote monitoring capability for healthcare providers; and alert notifications for device status and charging requirements.

### VI. Circumferential Bone Healing Monitoring

In yet another embodiment, a method and device for circumferential bone healing monitoring is disclosed. The monitoring may be achieved via the segmented articulated coil array, enabling spatial mapping of bone healing progression around the full circumference of an implant at the fracture site—a distinct and novel monitoring capability not achievable with conventional single-coil impedance measurement systems.

### A. Technical Background and Monitoring Gap

The OsteoCore platform's implanted passive electromagnetic components—comprising permanent magnets within a biocompatible cartridge integrated into orthopaedic fixation hardware —produce magnetic fields that radiate outward from the implant in a radial, circumferential pattern. This radial field distribution is inherent to the physics of the implanted magnet geometry: field energy projects outward from the implant surface in all radial directions, reaching the periosteal and endosteal bone healing surfaces circumferentially around the fracture site.

Accordingly, the therapeutic field delivery function of OsteoCore does not require a circumferential external coil—the implanted components themselves accomplish radially outward field concentration that addresses the full circumference of the healing bone.

However, a significant monitoring gap exists in current orthopaedic fracture management. Existing bone healing monitoring modalities—including serial radiography, CT scanning, and some Real-Time Monitoring (RTM) capabilities—provide only aggregate, spatially undifferentiated assessments of healing progress. A single external measurement coil positioned unilaterally over the fracture site produces a single impedance measurement representing a weighted average of tissue magnetic susceptibility changes across all periosteal surfaces simultaneously. This aggregate measurement cannot distinguish between symmetric healing—in which bone formation is progressing uniformly around the full implant circumference—and asymmetric healing, in which one quadrant of the periosteal surface is healing while the contralateral surface is lagging or failing to unite.

Asymmetric fracture healing is clinically significant, particularly in weight-bearing bones, comminuted fractures, and high-risk patients in whom partial non-union involving only one quadrant of the periosteal surface may be radiographically occult until mechanical failure occurs.

### B. Circumferential Bone Healing Monitoring via Segmented Articulated Coil Array

The segmented articulated coil array (Architecture C, FIG. 12, FIG. 13, FIG. 14, FIG. 15) is architecturally capable of functioning as a circumferential bone healing monitoring instrument when configured to encircle the anatomical region at the implant level. As illustrated in FIG. 14, the segmented articulated coil array achieves circumferential coverage ranging from approximately 100 degrees on large-circumference anatomies (55 cm thigh) to approximately 340 degrees on small-circumference anatomies (16 cm wrist). When the individually addressable coil elements of the array are used as impedance measurement coils—rather than, or in addition to, therapy delivery coils—each element independently measures the impedance of its local coupling to the implanted OsteoCore permanent magnets at its specific angular position around the body surface region.

Because the OsteoCore implanted magnets radiate their field outward in all radial directions from the implant surface, each coil element of the array—positioned at a distinct angular location—has a measurable magnetic coupling to the implanted magnets that is modulated by the tissue magnetic susceptibility of the healing zone in the radial sector between that coil element and the implant. As healing progresses in any given radial sector—transitioning from soft callus through mineralizing callus to mature bone—the tissue magnetic susceptibility in that sector changes, altering the impedance of the coil element overlying that sector. By independently measuring the impedance of each coil element, a processor can construct a spatial map of healing progression at each angular position, providing a circumferential healing profile that identifies both the overall stage of healing and the angular distribution of healing activity around the implant site.

In one embodiment, the circumferential monitoring protocol operates as follows: each coil element of the segmented array sequentially applies a low-amplitude interrogation signal and measures the resulting coil impedance, with the measurement sequence cycling through all elements to produce a complete angular impedance profile at each monitoring session (as illustrated in the sequential scanning mode of FIG. 15, Panel C). A magnetometer within the device confirms correct positioning of the array relative to the implanted magnets prior to each measurement cycle (FIG. 10). The angular impedance profile is processed by an onboard microcontroller and transmitted via Bluetooth to a paired smartphone application (FIG. 5), which displays a circumferential healing map showing impedance change—and therefore inferred healing stage—at each angular sector around the implant.

### C. Clinical Significance

Circumferential bone healing monitoring via the segmented articulated coil array addresses a clinically unmet need not satisfied by any currently available device. Clinical significance includes: (a) detection of asymmetric healing in large bone fractures (femur, tibia) where the compression and tension sides may heal at different rates; (b) spatial characterization of healing in comminuted fractures where multiple fracture lines are distributed around the bone circumference; (c) early identification of focal non-union at a specific angular sector before radiographic detection; and (d) quantitative longitudinal tracking of circumferential healing progression to guide return-to-activity decisions and implant removal timing. This circumferential monitoring capability is architecturally unique to the OsteoCore platform because it depends on the implanted passive permanent magnets as distributed passive sensors.

### VII. Stacked Permanent Magnet Polarity Configurations

Disclosed herein is a polarity configuration of stacked permanent magnets. The configurations may cover both alternating-pole and same-pole (non-alternating) arrangements and their respective field concentration mechanisms; static and mobile magnet configurations within the cartridge housing; and/or a single-magnet embodiment of any geometry as a distinct alternative to stacked multi-magnet configurations.

### A. Alternating-Pole Configuration

Permanent magnets may be arranged in a stacked configuration with alternating polarity—adjacent magnets oriented with opposite poles facing one another (N-S-N-S arrangement along the stack axis). In an alternating-pole configuration, each interface between adjacent magnets creates a magnetic field gradient in the region immediately surrounding the interface. The plurality of gradients produced along the stack creates a complex, multi-focal magnetic field pattern within and surrounding the cartridge housing, with radial gradient vectors extending outward from the cartridge into the surrounding bone and tissue.

### B. Non-Alternating (Same-Pole) Configuration

In some embodiments, stacked permanent magnets within the implantable passive electromagnetic component may be arranged in a non-alternating, same-pole configuration—adjacent magnets oriented with the same pole type facing one another at each interface (N-N or S-S arrangement), or all magnets oriented in the same sequential direction producing a reinforcing rather than opposing field at each interface.

The non-alternating configuration produces a different magnetic field geometry compared to the alternating-pole arrangement. In the same-directional arrangement, individual magnet fields sum constructively along the stack axis, producing a stronger axial magnetic field extending from the ends of the cartridge. This axially-concentrated field geometry may be advantageous in certain hardware configurations—for example, in an intramedullary nail where the cartridge is oriented with its long axis parallel to the long axis of the nail and bone. In the opposing same-pole configuration (N-N or S-S at the interface), the repulsive magnet geometry creates a strong radially-diverging field in the plane of the interface, projecting therapeutic field energy radially outward at the fracture level.

### C. Variable and Hybrid Configurations

The implantable passive electromagnetic component may further comprise combinations of alternating-pole and same-pole magnet arrangements within a single cartridge, creating hybrid field geometries tailored to specific fracture patterns and hardware configurations. In one embodiment, a cartridge designed for intramedullary nail use comprises a proximal alternating-pole zone and a distal same-pole zone, with the transition point positioned at the fracture plane. Furthermore, magnets within the cartridge housing may be moveable relative to one another, enabling modulation of the polarity configuration in response to externally applied magnetic fields, as disclosed in the parent application.

### D. Static and Mobile Magnet Configurations

The permanent magnet or magnets within the implantable passive electromagnetic component may be configured in either a static (fixed) or mobile configuration, or a combination thereof, within the cartridge housing. In a static configuration, the one or more permanent magnets are fixed within the housing and do not substantially move during implantation or use. In a mobile configuration, one or more permanent magnets are free to move within the housing—including freely floating, oscillating, or translating along the housing long axis—in response to patient motion, gravitational forces, or an externally applied magnetic field. Mobile magnet configurations may produce oscillating magnetic fields endogenously through patient ambulation, providing passive therapeutic field generation independent of any external energy source.

Hybrid configurations comprising both fixed and mobile magnets within the same housing are also contemplated, wherein fixed magnets establish a baseline static magnetic field geometry while mobile magnets superimpose a dynamic, motion-responsive field component.

### E. Single-Magnet Embodiment—Geometry-Neutral Coverage

The implantable passive electromagnetic component of the present disclosure may comprise a single permanent magnet of any geometry housed within the biocompatible cartridge. The single magnet may be fixed or mobile within the cartridge housing and may comprise neodymium, ceramic, samarium cobalt, AlNiCo, or any other biocompatible permanent magnet material, in any geometry.

### VIII. Dual-Modality Sequential Therapy Using the Compact PEMF Companion Device

Disclosed herein is dual-modality sequential therapy use of the compact PEMF companion device. The therapeutic use may include sequential treatment of bone healing. In some embodiments, the sequential treatment is used in conjunction with one or more additional therapeutic applications, such as biofilm disruption.

### A. Multi-Application Therapeutic Potential

The compact wearable PEMF generator may be architecturally capable of generating pulsed electromagnetic fields at selectable frequencies, waveforms, and amplitudes across a range of 1 Hz and above (including frequencies up to and exceeding 500 Hz) and field amplitudes of 3-20 mT. This parametric flexibility may enable the device to deliver therapeutic electromagnetic fields optimized for multiple distinct biological targets in discrete, sequentially programmed time periods within a single therapy session.

The pulsed electromagnetic fields may influence multiple biological processes simultaneously within exposed tissue, including: (a) osteoblast activation and bone matrix deposition; (b) disruption of bacterial biofilm integrity and cell membrane permeability; (c) promotion of angiogenesis and vascularization; (d) modulation of inflammatory cytokine expression; and (e) stimulation of chondrocyte activity for cartilage repair.

### B. Sequential Bone Healing and Biofilm Disruption Protocol

In some embodiments, the compact PEMF generator is configured to deliver electromagnetic therapy addressing both fracture healing and biofilm prevention or disruption within the same therapy session through a programmed sequential protocol. Fracture-related infection (FRI) is a major complication of orthopaedic trauma surgery, with infection rates of 1-5% in closed fractures and up to 30% in severe open fractures.

In one embodiment, the compact PEMF generator is programmed to deliver a sequential therapy protocol within each therapy session comprising: (a) a first therapy phase delivering a primary therapeutic frequency (e.g., 15-75 Hz) optimized for osteoblast activation and fracture healing, administered for a first prescribed time period; followed by (b) a second therapy phase delivering a secondary therapeutic frequency (e.g., 150-500 Hz) or modified waveform optimized for biofilm disruption and antimicrobial effect, administered for a second prescribed time period within the same session.

### C. Other Sequential Therapy Applications

The dual-modality sequential PEMF delivery architecture disclosed herein is not limited to bone healing and biofilm disruption. Additional sequential therapy combinations within the scope of the present disclosure include, without limitation: (a) fracture healing and osseointegration promotion; (b) fracture healing and periimplant pain control via electromagnetic neuromodulation; (c) fracture healing and prevention of periimplant avascular necrosis via PEMF-enhanced angiogenesis; (d) fracture healing and chondral repair in periarticular fractures; and/or (e) any other combination of therapeutic targets for which pulsed electromagnetic field stimulation may provide a biological effect. The sequential multi-phase protocol may be extended to three or more therapy phases within a single session, wherein each phase may be independently configured for a distinct therapeutic target.

### IX. Integration With Drug-eluting Orthopaedic Implants

Disclosed herein is use of OsteoCore passive electromagnetic components in conjunction with drug-eluting orthopaedic implants, including but not limited to antibiotic-eluting, anti-inflammatory-eluting, and growth factor-eluting hardware, and combinations thereof.

### A. Drug-eluting Orthopaedic Implants—Background

Drug-eluting orthopaedic implants are medical hardware devices that incorporate a pharmacological agent within or on their structure for controlled local release at the implantation site. Current drug-eluting orthopaedic implant technologies include: (a) antibiotic-eluting implants, including intramedullary nails with biodegradable polymer coatings containing gentamicin sulfate (e.g., Expert Tibial Nail PROtect, DePuy Synthes), and antibiotic-loaded calcium sulfate or calcium phosphate spacers; (b) anti-inflammatory-eluting implants; and (c) osteoinductive agent-eluting implants incorporating bone morphogenetic proteins (BMPs) or platelet-derived growth factors (PDGFs).

### B. Integration With Drug-eluting Implants

The OsteoCore passive electromagnetic component may be integrated within orthopaedic hardware that additionally comprises a drug-eluting surface coating or drug-loaded internal reservoir. Such integration may beneficially create a multi-modal therapeutic implant system combining electromagnetic field therapy and local pharmacological therapy at the implantation site. In one embodiment, an intramedullary nail comprises: (a) an OsteoCore passive electromagnetic cartridge inserted within the cannulated portion of the nail body at the level of the fracture, and (b) a drug-eluting surface coating on the exterior surface of the nail comprising a biodegradable polymer matrix incorporating a pharmacological agent.

### C. Mechanisms of Electromagnetic-Pharmacological Synergy

The combination of OsteoCore PEMF field concentration and local drug delivery produces therapeutic synergy through multiple mechanisms, including but not limited to: (a) Enhanced Antibiotic Efficacy—PEMF-induced increase in bacterial cell membrane permeability augments intracellular accumulation of aminoglycoside antibiotics; (b) Biofilm Matrix Disruption—PEMF fields disrupt the extracellular polysaccharide matrix of bacterial biofilm, exposing bacteria previously protected within the biofilm to locally delivered antibiotics; (c) Enhanced Tissue Penetration—electromagnetic fields may enhance transport of pharmacological agents through periimplant tissue; and/or (d) Concurrent Bone Healing Promotion—the OsteoCore PEMF field concentration simultaneously promotes osteoblast activity.

### D. Drug Classes and Delivery Systems

The integration with drug-eluting implants disclosed herein encompasses any pharmacological agent whose therapeutic activity, bioavailability, tissue penetration, cellular uptake, or mechanism of action is enhanced, augmented, or modulated by externally applied or locally concentrated pulsed electromagnetic fields. By way of non-limiting example, drug-eluting configurations include: (a) antibiotic-eluting configurations comprising aminoglycosides, glycopeptides, beta-lactams, rifampin, or fluoroquinolones; (b) anti-inflammatory agent-eluting configurations; (c) osteoinductive and bone anabolic agent-eluting configurations comprising BMPs, PDGFs, parathyroid hormone-related peptide analogs, or sclerostin inhibitors; (d) anti-biofilm agent-eluting configurations; (e) angiogenic agent-eluting configurations, and/or (f) combinations thereof.

### X. Switchable Permanent Magnet Embodiments and Broad Medical-Grade Magnet Material Coverage

Disclosed herein are switchable permanent magnet embodiments comprising low-coercivity magnetic alloys. The alloys may include, but are not limited to, aluminum-nickel-cobalt (AlNiCo) alloys and all other medical-grade permanent magnet materials. The switchable embodiments may be configurable between a magnetized therapeutic state and a demagnetized imaging state, which may enable artifact-minimized post-operative CT and MRI imaging without surgical removal of the implanted component.

### A. Medical-Grade Permanent Magnet Material Coverage

The implantable passive electromagnetic components of the present disclosure are not limited to any particular permanent magnet material. The present disclosure expressly encompasses all biocompatible permanent magnet materials suitable for implantation within orthopaedic hardware, including without limitation: neodymium-iron-boron (NdFeB) alloys; ceramic (ferrite) magnets; samarium-cobalt (SmCo) alloys; aluminum-nickel-cobalt (AlNiCo) alloys; any other permanent magnet material that is biocompatible when encapsulated within a biocompatible housing as disclosed herein; and/or combinations thereof.

### B. Switchable Permanent Magnet Embodiments

A clinically significant limitation of high-coercivity permanent magnet implants—including NdFeB alloys—is the generation of magnetic susceptibility artifact in post-operative imaging modalities, including CT and MRI. The strong, stable magnetization of high-coercivity NdFeB magnets produces metal artifact that can substantially degrade image quality at and around the fracture site. There exists a clinically significant unmet need for an implantable permanent magnet components that can be selectively demagnetized prior to post-operative imaging to minimize or eliminate imaging artifact, and subsequently re-magnetized following completion of imaging to restore full therapeutic function.

### C. Low-coercivity Switchable Magnet Materials

In one embodiment, aluminum-nickel-cobalt (AlNiCo) alloys are used as the switchable permanent magnet materials for this application. AlNiCo alloys exhibit coercivity values in the range of approximately 40 to 160 kA/m—substantially lower than the coercivity of NdFeB alloys (approximately 900 to 2,000 kA/m). This low coercivity enables demagnetization of an implanted AlNiCo magnet by application of an external decaying alternating magnetic field of approximately 150 to 400 kA/m initial amplitude, which is achievable using external electromagnetic coil devices of clinically practical size and power consumption. The demagnetization protocol uses a decaying alternating field—not a single DC opposing pulse—to progressively reduce net magnetization toward zero through successive diminishing field reversals, thereby achieving a substantially demagnetized state. Following imaging, a re-magnetizing field of similar magnitude may be used to restore the AlNiCo magnet to its pre-imaging remanent magnetization.

### D. Demagnetization and Re-Magnetization Protocols

In one embodiment, a demagnetization protocol for an implanted switchable permanent magnet component comprises positioning an external electromagnetic coil device over the implant site and delivering a decaying alternating magnetic field of sufficient initial amplitude to exceed the coercive field of the implanted magnet material. The decaying envelope of the alternating field progressively reduces the applied field amplitude toward zero, causing the net magnetization of the magnet to converge toward zero remanent magnetization. In a further embodiment, the compact wearable PEMF companion device is configured to execute a demagnetization protocol. This may be achieved via a dedicated demagnetization operating mode, selectable via the device interface or paired smartphone application (FIG. 5), with magnetometer-based confirmation (FIG. 10) that implant magnetization has been reduced below the imaging artifact threshold. A re-magnetization protocol may be executed following completion of imaging by applying a unidirectional magnetizing field of sufficient amplitude in the correct polarity orientation.

### E. Hybrid Magnet Stack Configurations

In embodiments comprising stacked multi-magnet configurations, each magnet within the stack may independently comprise a switchable low-coercivity alloy. Alternatively, hybrid configurations comprising both high-coercivity NdFeB magnets and low-coercivity switchable magnets within the same cartridge are contemplated. In some hybrid stack embodiments, the switchable magnet component is positioned specifically at the level of the fracture gap—the anatomical region of greatest imaging interest and greatest therapeutic relevance—while NdFeB components are positioned at adjacent levels, maximizing imaging artifact reduction at the fracture gap while preserving NdFeB-derived therapeutic field strength at adjacent bone levels.

### XI. Spinal Fusion and Axial Skeletal Applications

Disclosed herein are spinal fusion and axial skeletal applications. These applications may cover OsteoCore passive electromagnetic component integration into spinal fusion hardware for cervical, thoracic, lumbar, and sacral fusion procedures, with paraspinal and cervical placement of the compact PEMF generator.

### A. Clinical Background—Spinal Fusion and Pseudarthrosis

Spinal fusion likely represents one of the largest and most clinically significant applications for the OsteoCore electromagnetic therapy platform. Approximately 460,000 spinal fusion procedures are performed annually in the United States, encompassing cervical, thoracic, lumbar, and sacral levels. Despite advances in surgical technique, bone graft materials, and fixation hardware, pseudarthrosis —failed spinal fusion —remains a major complication, occurring in 5-35% of cases depending on procedure type, patient risk factors, and spinal level. Pseudarthrosis is associated with persistent pain, hardware failure, and the need for revision surgery.

The EBI SpinalPak (CCEF) has FDA clearance specifically for adjunctive treatment of spinal fusion—establishing regulatory precedent for electromagnetic stimulation in the spinal fusion context. However, the SpinalPak requires implanted active electronics and battery removal surgery, and existing external PEMF devices are not designed for the geometric and anatomical requirements of paraspinal delivery. The OsteoCore platform—comprising a passive electromagnetic cartridge implanted directly within hardware (e.g., spinal fusion hardware), activated by the compact external PEMF generator positioned on a surface (e.g., the paraspinal or cervical surface)—eliminates all implanted active electronics while delivering concentrated electromagnetic therapy precisely at the fusion site.

### B. Cartridge Integration Into Spinal Fusion Hardware

The OsteoCore passive electromagnetic cartridge is configurable for integration into a broad range of spinal fusion hardware types. The biocompatible cartridge housing may be sized and shaped for integration into the following spinal fixation devices, by way of non-limiting example:
Interbody fusion cages (TLIF, PLIF, ALIF, XLIF, OLIF configurations): The cartridge is integrated within the hollow interior of the fusion cage body, positioned to deliver electromagnetic fields directly to the bone graft material and endplate surfaces within the cage. The cartridge may be pre-loaded into the cage prior to implantation or inserted into the cage via a loading port after cage implantation.
Pedicle screws: The cartridge is integrated within the cannulated bore of a cannulated pedicle screw, positioned within the vertebral body to deliver electromagnetic fields to the pedicle-vertebral body junction and adjacent cancellous bone.
Vertebral body implants and expandable cages: The cartridge is integrated within the structural body of the vertebral body replacement implant, positioned at the implant-endplate interface to promote fusion at the superior and inferior endplate surfaces.
Posterior spinal fixation rods and connectors: The cartridge is integrated within hollow rod segments positioned at the fusion level, delivering electromagnetic fields to the posterolateral fusion mass.
Anterior cervical discectomy and fusion (ACDF) plates and cages: The cartridge is integrated within the ACDF cage body or anterior plate construct, positioned to deliver electromagnetic fields to the interbody graft and endplate surfaces at the cervical fusion level.

In some spinal fusion hardware configurations, the cartridge comprises permanent magnets in a biocompatible PTFE, titanium, or PEEK housing, and may function as a passive electromagnetic concentrator that amplifies and focuses the externally applied PEMF field at the fusion site without requiring implanted batteries, active electronics, or transcutaneous wiring.

### C. External Pemf Generator Placement for Spinal Applications

The compact external PEMF generator is adapted for paraspinal and cervical placement in spinal fusion applications. Unlike extremity fracture applications where the generator is positioned circumferentially around a limb, spinal applications require unilateral or bilateral placement of the external generator on the patient's back, lumbar region, or neck overlying the fusion level. The conformable coil architectures—the curved rigid coil (Architecture A, FIG. 12, Panel A), the flexible continuous coil on flex-PCB substrate (Architecture B, FIG. 12, Panel B), and the segmented articulated coil array (Architecture C, FIG. 12, Panel C)—are each adapted for paraspinal placement as follows:

For lumbar and thoracic spinal fusion: The segmented articulated coil array or flexible continuous coil is positioned on the patient's back overlying the paraspinal musculature at the fusion level. The conformable substrate adapts to the contour of the paraspinal surface—a broad, gently curved anatomical region—enabling consistent therapeutic positioning across patients with varying body habitus. The generator may be integrated into a lumbar support garment or posterior trunk orthosis, analogous to the textile compression garment integration disclosed for extremity applications (FIG. 16), with a dedicated posterior placement pocket positioning the conformable PEMF device against the paraspinal skin surface at the fusion level.

For cervical spinal fusion: The compact disc-shaped PEMF generator (such as that illustrated in FIG. 3, FIG. 4) or a specially configured cervical conformable coil is positioned on the posterior or anterolateral neck overlying the cervical fusion level. The cervical anatomy presents a partially circumferential geometry, and the curved rigid coil architecture or a cervical collar-integrated conformable coil may provide optimal field delivery geometry for cervical applications. The compact generator mass (200-1200 grams) and attachment mechanism are adapted for cervical use via a cervical collar or neck brace integration.

### D. Field Delivery Geometry in Spinal Applications

In spinal fusion applications, the OsteoCore implanted cartridge within the fusion cage or pedicle screw may concentrate the externally applied PEMF field at the fusion site through a passive magnetic concentrator mechanism, such as that illustrated in FIG. 6. The external compact PEMF generator delivers a pulsed magnetic field to the paraspinal surface (in implant-activation mode: 3-10 mT external field output; in standalone mode: 8-20 mT). The implanted passive cartridge concentrates this external field at the intravertebral or interbody fusion site, eliminating the geometric field divergence that would otherwise attenuate field strength between the paraspinal skin surface and the deep spinal fusion hardware.

The bone healing monitoring system (such as that illustrated in FIG. 10, FIG. 11) may also be adapted for spinal fusion monitoring using the same impedance measurement principle. Impedance changes in the external measurement coil resulting from tissue magnetic susceptibility changes at the fusion site—as the bone graft progresses from fibrovascular incorporation through bony bridging to solid fusion—provide a non-invasive indicator of fusion progress, analogous to fracture healing monitoring capability.

### E. Applicable Spinal Fusion Indications

The OsteoCore spinal fusion platform may be applicable to spinal fusion indications in which electromagnetic field therapy at the fusion site may accelerate or improve fusion rates, including without limitation: (a) degenerative disc disease at any spinal level (cervical, thoracic, lumbar, sacral); (b) spinal stenosis with instability; (c) spondylolisthesis (all grades); (d) post-traumatic spinal instability and fracture-dislocation; (e) spinal deformity correction (scoliosis, kyphosis) with instrumented fusion; (f) adjacent segment disease following prior fusion; (g) revision fusion surgery for pseudarthrosis; (h) oncologic spinal reconstruction following tumor resection; and/or (i) infection-related spinal instability requiring fusion after debridement. The dual-modality sequential therapy capability described earlier herein may be particularly relevant in spinal fusion applications where periimplant infection and biofilm formation on spinal hardware represent major complications.

### XII. Craniofacial Skeletal and Aesthetic Applications

Disclosed herein are craniofacial and aesthetic applications. These applications may comprise (a) implantable OsteoCore components integrated into facial fracture fixation hardware for craniofacial skeletal healing, and/or (b) external PEMF delivery to facial dermal and subdermal tissue. These applications may find use for cosmetic skin rejuvenation, post-surgical facial edema reduction, and radiodermatitis treatment. In some embodiments, a facial mask may comprise a conformable external PEMF generator, which may be in the form of a conformable coil architecture.

### A. Overview—Dual Craniofacial Application Architecture

The OsteoCore platform may support two distinct but complementary applications in the craniofacial region, each representing a separate and independently valuable embodiment of the energy delivery system: (1) a skeletal medical application in which OsteoCore passive electromagnetic cartridges are integrated into craniofacial fracture fixation hardware to accelerate healing of facial bone fractures—a fracture healing application for the craniofacial skeleton; and (2) an aesthetic and therapeutic application in which the conformable external PEMF generator—in, for example, a facial mask form—delivers pulsed electromagnetic fields to facial dermal and subdermal tissue, which may beneficially impact skin rejuvenation, post-surgical edema reduction, and/or radiodermatitis treatment.

These two applications may be employed independently or in combination. For example, in a patient who has undergone open reduction and internal fixation (ORIF) of a facial fracture with OsteoCore-enabled hardware, the facial mask PEMF device may simultaneously serve both applications—activating the implanted cartridge at the fracture site for skeletal healing while delivering standalone PEMF to the surrounding facial soft tissue for edema reduction and tissue recovery.

### B. Craniofacial Fracture Fixation—Skeletal Application

Craniofacial fractures—including orbital floor fractures, zygomatic fractures, mandibular fractures, maxillary (Le Fort) fractures, nasal fractures, and frontal sinus fractures—are treated with open reduction and internal fixation (ORIF) using titanium plates, screws, and mesh systems. The healing of craniofacial fractures involves the same biological mechanisms as appendicular fracture healing: osteoblast activation, callus formation, mineralization, and remodeling. Accordingly, the disclosed platform herein may be used for craniofacial fracture healing, particularly via the disclosed electromagnetic bone healing mechanisms.

In one embodiment, the passive electromagnetic cartridge is integrated within craniofacial fixation hardware, including without limitation:
Titanium mini-plates and micro-plates used for mandibular, zygomatic, orbital, and frontal fracture fixation: The cartridge is integrated within the plate body or incorporated into a modified plate design with a housing for the passive electromagnetic component, positioned to deliver electromagnetic fields to the fracture site.
Orbital floor implants (titanium mesh or resorbable plate): The cartridge is integrated within or affixed to the orbital floor implant, delivering electromagnetic fields to the orbital floor fracture healing zone.
Mandibular reconstruction plates: For mandibular fractures or oncologic reconstruction, the cartridge within the reconstruction plate delivers concentrated PEMF to the osteotomy or fracture site.
Craniofacial fixation screws: Cannulated self-tapping screws used in craniofacial ORIF may incorporate the passive cartridge in the cannulated bore, analogous to the disclosed bone screw embodiment.

When the OsteoCore craniofacial implant is activated by the facial mask PEMF device (described in Section XII. C), the external facial mask generator delivers therapeutic PEMF to the facial surface overlying the fracture site, and the implanted passive cartridge concentrates this external field at the fracture line—providing the same geometric field convergence advantage at craniofacial fracture sites that the OsteoCore cartridge provides at long bone fracture sites.

The proximity of the craniofacial skeleton to the facial skin surface represents a particular advantage for the OsteoCore platform in this application. In long bone fractures, the external PEMF generator must deliver field energy through several centimeters of soft tissue to reach the implanted cartridge. In craniofacial fractures, the distance between the skin surface and the underlying fixation hardware is typically 2-5 mm, substantially reducing the field attenuation challenge and enabling even lower external field strengths to effectively activate the implanted passive concentrator.

### C. Facial Mask PEMF Device—Aesthetic and Therapeutic Application

In a further embodiment, the conformable coil architecture is configured as a facial mask. The mask may be used for delivery of pulsed electromagnetic fields to facial dermal and subdermal tissue. The facial mask embodiment may represent an adaptation of the segmented articulated coil array (Architecture C, FIG. 12, FIG. 13, FIG. 15) and flexible continuous coil (Architecture B) to the anatomical geometry of at least a portion of a human face, may be configured for standalone PEMF delivery to facial soft tissue independent of any implanted components, and/or for combined implant activation and soft tissue PEMF delivery in craniofacial fracture patients.

### C.1. Facial Mask and Architecture

In facial mask embodiments, the PEMF device may comprise a conformable substrate shaped and sized to conform to at least a portion of a human face, including but not limited to:
A continuous or segmented coil substrate configured to cover the major facial anatomical regions including the frontal/temporal zone, periorbital zone, malar/zygomatic zone, nasolabial zone, mandibular zone, and optional cervical extension for submandibular and neck coverage.
Anatomical apertures for the eyes, nose, and mouth, enabling sustained wear during treatment sessions without obstruction of vision, breathing, or verbal communication. Aperture geometry is sized to accommodate normal facial anthropometric variation.
A conformable substrate material comprising flexible PCB substrate, silicone elastomer, or other biocompatible flexible material enabling conformal contact with the curved facial surface across the range of normal facial geometries.
An attachment system comprising adjustable straps, magnetic closures, or other retention means configured to maintain consistent therapeutic positioning of the device against the facial surface during treatment sessions of 15-60 minutes.
A central electronics module—equivalent to the electronics module disclosed for the extremity segmented array (FIG. 13)—may integrated into the mask structure or connected via flexible leads to a separate wearable electronics unit, which may comprise a microcontroller, pulse generation circuit, rechargeable battery, and/or Bluetooth communication module (FIG. 5, FIG. 8).

### C.2. Facial Anatomical Zone Mapping and Selective Activation

Analogous to the selective coil element activation disclosed for the extremity segmented array (FIG. 15), the facial mask embodiment including a PEMF device may support independent addressability of facial anatomical zones, enabling selective activation of specific regions within a single treatment session. Facial zone addressability supports, but is not limited to, the following:
Targeted periorbital therapy for post-blepharoplasty edema reduction, periorbital wrinkle treatment, and orbital floor fracture healing.
Malar zone therapy for post-facelift recovery, zygomatic fracture healing, and malar soft tissue rejuvenation.
Mandibular zone therapy for mandibular fracture healing and jawline contouring.
Full-face treatment for anti-aging, collagen stimulation, and radiodermatitis management.

Zone-specific treatment parameters—frequency, pulse width, field amplitude, and session duration—may be independently programmed for each facial zone, including for example via the paired smartphone application (FIG. 5), enabling clinician-prescribed or protocol-driven facial treatment regimens.

### C.3. Biological Mechanisms—Facial Dermal and Subdermal PEMF Effects

The biological rationale for facial PEMF application may produce the following effects relevant to facial aesthetic and therapeutic applications:
Collagen synthesis stimulation: PEMF increases production of collagen fibers by dermal fibroblasts through non-thermal mechanisms, as established in multiple in vitro and in vivo studies. Increased dermal collagen density improves skin elasticity, reduces rhytide depth, and improves overall skin quality.
Angiogenesis: PEMF stimulates endothelial cell proliferation and angiogenesis through endothelial release of FGF-2, increasing dermal vascularity and improving tissue oxygenation and nutrient delivery.
Anti-inflammatory effects: PEMF suppresses inflammatory cytokine expression via adenosine A2A receptor activation and NF-κB pathway modulation, reducing post-surgical and post-procedural inflammation and edema.
Post-surgical edema reduction: PEMF therapy reduces post-operative soft tissue edema and pain following surgical procedures, including blepharoplasty and facelift surgery.
Radiodermatitis treatment: PEMF therapy significantly reduces post-radiation skin thickness and severity of acute radiodermatitis compared to conventional skin care alone, directly supporting facial PEMF application in oncologic patients receiving head and neck radiation therapy.

### C.4. Tissue Response Monitoring—Dermal RTM Analog

In a further embodiment, the bone healing monitoring system (such as that illustrated in FIG. 10, FIG. 11) is extended to a dermal tissue response monitoring (dRTM) capability using a facial mask embodiment described earlier herein. When OsteoCore passive components are implanted in craniofacial fixation hardware, the impedance measurement capability of the external coil may be used to monitor healing at the craniofacial fracture site through the facial skin surface, providing the same fracture healing staging capability described for appendicular fractures.

Additionally, in standalone aesthetic applications without implanted components, progressive changes in dermal tissue properties during a course of PEMF facial treatment—including increasing collagen density and improving tissue hydration—produce measurable changes in the electrical properties of the dermal tissue layer. These changes may be detectable as changes in the impedance characteristics of the facial mask coil over a treatment course, providing a non-invasive indicator of treatment response that can guide treatment duration and parameter optimization. This aesthetic tissue monitoring capability represents a novel extension of the RTM concept from bone healing to soft tissue remodeling.

### D. Regulatory Pathway Considerations

The facial mask PEMF device disclosed herein has two potential regulatory pathways depending on the intended indication: (a) for craniofacial fracture healing applications with implanted OsteoCore components, the regulatory pathway follows the same De Novo/510(k) strategy as the appendicular fracture healing system; (b) for standalone aesthetic applications (wrinkle reduction, skin rejuvenation), a 510(k) pathway utilizing the Venus Concept DiamondPolar/OctiPolar FDA clearance for non-invasive treatment of facial wrinkles and rhytides as a predicate is available. The Venus Concept clearance establishes that RF+PEMF combination devices have an FDA-recognized predicate for facial wrinkle treatment; the OsteoCore facial mask embodiment that delivers PEMF without RF is distinguishable from the predicate by its passive concentrator architecture and absence of thermal energy delivery, but the cleared indication provides a regulatory framework for the clinical claim.

What is claimed is:

1.-45. (canceled)

46. An implantable passive electromagnetic component for use in orthopaedic hardware, comprising:
(a) a biocompatible housing sized for integration within standard orthopaedic fixation hardware;
(b) one or more permanent magnets within said housing comprising a magnetic alloy having a coercivity between 10 kA/m and 500 kA/m, wherein said coercivity is sufficiently low to permit demagnetization and re-magnetization of said magnets by an externally applied decaying alternating magnetic field without surgical removal of said component;
(c) wherein said one or more magnets are configurable between a magnetized therapeutic state, in which said magnets concentrate externally applied pulsed electromagnetic fields at a bone or fusion site, and a demagnetized imaging state, in which net magnetization of said component is reduced to minimize magnetic susceptibility artifact in post-operative imaging modalities including computed tomography (CT) and magnetic resonance imaging (MRI); and
(d) wherein transition between said magnetized therapeutic state and said demagnetized imaging state is accomplished by application of an external decaying alternating magnetic field from a device external to the patient, without surgical intervention.

47. The implantable passive electromagnetic component of claim 46, wherein said magnetic alloy is an aluminum-nickel-cobalt (AlNiCo) alloy.

48. The implantable passive electromagnetic component of claim 46, wherein said magnetic alloy has a coercivity between 40 kA/m and 160 kA/m.

49. The implantable passive electromagnetic component of claim 47, wherein said AlNiCo alloy is selected from the group consisting of AlNiCo 2, AlNiCo 5, AlNiCo 8, and combinations thereof.

50. The implantable passive electromagnetic component of claim 46, further comprising one or more additional permanent magnets within said housing comprising a high-coercivity magnetic alloy having a coercivity greater than 500 kA/m, wherein said high-coercivity magnets maintain a baseline residual magnetic field at the treatment site during the demagnetized imaging state of said switchable magnets, providing therapeutic field continuity during imaging intervals.

51. The implantable passive electromagnetic component of claim 50, wherein said high-coercivity magnetic alloy comprises neodymium-iron-boron (NdFeB), and wherein said switchable low-coercivity magnets and said high-coercivity NdFeB magnets are arranged in a stacked configuration within said biocompatible housing.

52. The implantable passive electromagnetic component of claim 51, wherein said switchable low-coercivity magnets are positioned within said stacked configuration at a level corresponding to a bone fracture gap or fusion site, and wherein said high-coercivity NdFeB magnets are positioned at levels adjacent to said fracture gap or fusion site, thereby maximizing imaging artifact reduction at the treatment site while preserving therapeutic field strength at adjacent bone levels.

53.-59 (canceled)

60. An electromagnetic therapy system for spinal fusion, comprising:
(a) an implantable passive electromagnetic component configured for integration within spinal fusion hardware at a spinal fusion site, said component comprising one or more permanent magnets housed within a biocompatible encapsulation;
(b) a compact external pulsed electromagnetic field (PEMF) generator configured for positioning on a body surface region overlying said spinal fusion site, said generator comprising an electromagnetic coil, a pulse generation circuit, a rechargeable power source, and a wearable housing;
(c) wherein said external PEMF generator is configured to deliver a pulsed magnetic field to said implantable passive electromagnetic component through the patient's skin and paraspinal tissue; and
(d) wherein said implantable passive electromagnetic component concentrates said pulsed magnetic field at the spinal fusion site, eliminating geometric field divergence between the external generator and the fusion site.

61. The electromagnetic therapy system of claim 60, wherein said spinal fusion hardware is selected from the group consisting of: interbody fusion cages (TLIF, PLIF, ALIF, XLIF, and OLIF configurations), pedicle screws, vertebral body implants, expandable corpectomy cages, anterior cervical discectomy and fusion (ACDF) cages and plates, posterior spinal fixation rods, spinal interbody spacers, and combinations thereof.

62. The electromagnetic therapy system of claim 60, wherein said compact external PEMF generator is configured for paraspinal placement on the patient's back overlying a lumbar or thoracic fusion level, and wherein said generator is integrated into or secured by a posterior trunk orthosis, lumbar support garment, or paraspinal positioning harness configured to maintain consistent therapeutic positioning of the generator over the fusion site during normal daily activities.

63. The electromagnetic therapy system of claim 60, wherein said compact external PEMF generator is configured for cervical placement on the patient's posterior or anterolateral neck overlying a cervical fusion level, and wherein said generator is integrated into or secured by a cervical collar or neck brace.

64. The electromagnetic therapy system of claim 60, wherein said conformable external PEMF generator comprises a conformable substrate configured to conform to a paraspinal body surface region, said conformable substrate selected from: a curved rigid coil having a concave patient-contact surface; a flexible continuous coil on a flex-PCB substrate; and a segmented articulated coil array comprising a plurality of discrete coil elements connected by flexible conductors.

65. The electromagnetic therapy system of claim 60, further comprising a bone fusion monitoring system configured to non-invasively assess spinal fusion progress, wherein impedance of the external PEMF generator coil is measured and changes in said impedance resulting from tissue magnetic susceptibility changes at the fusion site are used to classify fusion stage.

66. The electromagnetic therapy system of claim 60, wherein said external PEMF generator is further configured to deliver a sequential multi-phase therapy protocol comprising: (a) a first therapy phase at a first frequency optimized for osteoblast activation and bone graft incorporation; and (b) a second therapy phase at a second frequency optimized for biofilm disruption and infection prophylaxis at the spinal implant surface.

67. The electromagnetic therapy system of claim 60, wherein said implantable passive electromagnetic component is integrated within an interbody fusion cage positioned at a spinal fusion level, wherein said component is pre-loaded within the hollow interior of said cage prior to implantation or inserted via a loading port after cage implantation.

68.-83. (canceled)

84. A method of delivering pulsed electromagnetic field therapy to the face, comprising:
(a) positioning a conformable facial PEMF device against the patient's facial surface, said device comprising a conformable substrate with anatomical apertures for the eyes, nose, and mouth, and a plurality of electromagnetic coil elements distributed across facial anatomical zones;
(b) securing said device against the facial surface via an attachment mechanism;
(c) activating said device to generate pulsed electromagnetic fields at one or more facial anatomical zones; and
(d) wherein said pulsed electromagnetic fields are delivered to facial dermal and subdermal tissue to achieve at least one of: collagen synthesis stimulation, angiogenesis promotion, anti-inflammatory effect, post-surgical edema reduction, skin laxity improvement, rhytide reduction, and radiodermatitis treatment.

85. The method of claim 84, wherein said method further comprises implant-activation of passive electromagnetic components within craniofacial fixation hardware, wherein said facial PEMF device simultaneously delivers pulsed magnetic fields to activate said implanted passive components at a craniofacial fracture site and to the surrounding facial soft tissue for edema reduction and tissue recovery.

86. The method of claim 84, wherein said pulsed electromagnetic fields are delivered to the periorbital zone for treatment of post-blepharoplasty edema, periorbital rhytides, or orbital floor fracture healing.

87. The method of claim 84, wherein said pulsed electromagnetic fields are delivered to facial skin affected by radiodermatitis resulting from head and neck radiation therapy, wherein said fields reduce skin thickness and severity of acute radiodermatitis.

88. The method of claim 84, wherein said device is used in combination with topical skincare agents, wherein said pulsed electromagnetic fields transiently increase dermal permeability to enhance penetration of said topical agents into dermal tissue.

89. (canceled)
